(19) United States
(12) Patent Application Publication
(10) Pub. No.: US 2012/0171729 A1
(43) Pub. Date: Jul. 5, 2012

(54) METHODS FOR THE SYNTHESIS OF POLYADENYLIC ACID

(75) Inventors: Mark W. Eshoo, Solana Beach, CA (US); Curtis Phillipson, Carlsbad, CA (US)

(73) Assignee: IBIS BIOSCIENCES, INC., Carlsbad, CA (US)

(21) Appl. No.: 13/340,962

(22) Filed: Dec. 30, 2011

Related U.S. Application Data
(60) Provisional application No. 61/428,394, filed on Dec. 30, 2010.

(51) Int. Cl. C12P 19/34; C12N 9/10

(52) U.S. Cl. 435/91.5; 435/193

(57) ABSTRACT

The present invention provides compositions, kits, and methods for synthesizing polyadenylic acid using polynucleotide phosphorylase, adenosine diphosphate, a buffering agent, and a divalent metal cation. In certain embodiments, the adenosine diphosphate is present at a concentration between about 5.0 mM and about 100 mM, and the buffering agent has a pH around 8.0. In some embodiments, the prepared polyadenylic acid compositions are free of detectable nucleic acid.

The present Application claims priority to U.S. Provisional Application Ser. No. 61/428,394 filed Dec. 30, 2010, the entirety of which is incorporated by reference herein.

### FIELD OF THE INVENTION

The present invention provides compositions, kits, and methods for synthesizing polyadenylic acid using polynucleotide phosphorylase, adenosine diphosphate, a buffering agent, and a divalent metal cation. In certain embodiments, the adenosine diphosphate is present at a concentration between about 5.0 mM and about 100 mM, and the buffering agent has a pH around 8.0. In some embodiments, the prepared polyadenylic acid compositions are free of detectable contaminating nucleic acid.

### BACKGROUND

Polyadenylic acid is a polymer of adenylic acid that is sometimes attached to eukaryotic messenger RNA and stabilizes the molecule before transport from the nucleus into the cytoplasm. Poly A is used in the biotechnology industry in various buffers and as a carrier in DNA and RNA extractions. Commercial preparations of nucleic acid-free preparations of polyadenylic acid are not currently available.

### SUMMARY OF THE INVENTION

The present invention provides compositions, kits, and methods for synthesizing polyadenylic acid using polynucleotide phosphorylase, adenosine diphosphate, a buffering agent, and a divalent metal cation. In certain embodiments, the adenosine diphosphate is present at a concentration between 5.0 mM and 100 mM, and the buffering agent has a pH between 7.3 and 8.7. In some embodiments, the prepared polyadenylic acid compositions are free of detectable contaminating nucleic acid.

In some embodiments, the present invention provides compositions comprising, or consisting essentially of, or consisting of: a) polynucleotide phosphorylase; b) adenosine diphosphate (ADP) (e.g., present at a concentration between 5.0 mM and 100 mM); c) a buffering agent (e.g., with a pH between 7.3 and 8.7; or about 8.0); and d) a divalent metal cation.

In particular embodiments, the present invention provides systems or kits comprising or consisting essentially of the following components: a) polynucleotide phosphorylase; b) adenosine diphosphate (ADP) (e.g., present at a concentration between 5.0 mM and 100 mM); c) a buffering agent (e.g., with a pH between 7.3 and 8.7); and d) a divalent metal cation.

In other embodiments, the present invention provides methods of making polyadenylic acid comprising: a) combining polynucleotide phosphorylase, adenosine diphosphate (ADP), a buffering agent (e.g., with pH between 7.3 and 8.7), and a divalent metal cation, to generate a mixture (e.g., wherein the ADP is present in the mixture at a concentration between 5.0 mM and 100 mM); and b) incubating the mixture under conditions such that a composition comprising polyadenylic acid is generated.

In particular embodiments, the incubating is conducted for 30 minutes to 150 hours (e.g., 30 minutes . . . 1 hour . . . 5 hours . . . 30 hours . . . 50 hours . . . 75 hours . . . 100 hours . . . 125 hours . . . 150 hours). In some embodiments, the incubating is conducted for 100 hours to 125 hours (e.g., 100 . . . 110 . . . 115 . . . 120 . . . or 125 hours). In certain embodiments, the incubating is conducted at a temperature between 30 and 50 degrees Celsius (e.g., 30 . . . 35 . . . 40 . . . 45 . . . or 50 degrees Celsius). In other embodiments, the incubating is conducted at a temperature between 38 and 46 degrees Celsius.

In particular embodiments, the compositions are free of detectable nucleic acid (e.g., no nucleic acid could be visualized by standard gel electrophoresis). In other embodiments, the pH is between 7.7 and 8.3 (e.g., 7.7, 7.8, 7.9, 8.0, 8.1, 8.2, or 8.3).

In further embodiments, the pH is between 7.9 and 8.1.

In some embodiments, the ADP is present at a concentration between 10.0 mM and 75 mM (e.g., 10 mM . . . 20 mM . . . 30 mM . . . 50 mM . . . 60 mM . . . or 75 mM). In particular embodiments, the ADP is present at a concentration between 20.0 mM and 50 mM. In certain embodiments, the divalent metal cation is MgCl₂.

### DESCRIPTION OF THE FIGURES

FIG. 1 shows that condition from the prior art reference of Jones and Bibb produced no poly-A as visualized using gel electrophoresis.

FIG. 2 shows a gel from Example 2 that shows that a change to a pH of 8.0 was able to generate polyadenylic acid.

FIG. 3 shows a gel that shows that the synthesis reaction from Example 2 could be inhibited by high concentrations of ADP and that shorter incubation times generated higher molecular weight poly-A, and longer incubation times generated lower molecular weight poly-A.

### DETAILED DESCRIPTION

The present invention provides compositions, kits, and methods for synthesizing polyadenylic acid using polynucleotide phosphorylase, adenosine diphosphate, a buffering agent, and a divalent metal cation. In certain embodiments, the adenosine diphosphate is present at a concentration between 5.0 mM and 100 mM, and the buffering agent has a pH between 7.4 and 8.6. In some embodiments, the prepared polyadenylic acid compositions are free of detectable contaminating nucleic acid.

The polyadenylic acid produced according the methods of the present invention may be used, for example, in many biotechnology applications such as in various buffers and as a carrier in DNA and RNA extractions.

### EXAMPLES

### Example 1

### Method of Making Poly-A

This Example describes an exemplary method for making polyadenylic acid using an IV bag. First, each 1 g vial of ADP used is reconstituted with 4 ml of water to bring it up to a 250 mg/ml solution. Next, the following amounts of reagents are injected into a 500 ml IV bag to produce a reaction buffer: 20 ml of 250 mg/ml ADP, 25 ml of 1M Tris, pH 8.0, 5 ml of 1M MgCl₂, and 450 ml of Water. The solution is then gently mixed by swirling the bag. The solution can then be passed through a filter (e.g., a 0.2 um filter) and transferred to a new IV bag, while discarding the first 50 ml of the filtrate. A syringe is then used to inject an amount of polynucleotide phosphorylase equivalent to 189 Us. The solution is then mixed gently on a rocker for about one hour at room temperature. The solution is then incubated at 42C for about 120 hours. The solution is mixed daily by gently inverting the bag three times. This method will generate polyadenylic acid that is free from contaminating nucleic acid.

The polyadenylic acid generated may be tested and recovered as follows. A small amount of the final solution (e.g., 0.5 ml) may be run on a 1% agarose gel to ensure that the reaction has gone to completion. Next, obtain 2 Amicon filters and to each one add 15,000 ul of 2M KCl. Centrifuge tubes for 10 minutes at 3000 rpm. Carefully pool KCl filtrate into a fresh, UV-treated 50 ml conical tube. Using a syringe, remove approximately 22.7 ml of poly-A solution from the IV bag and pour into a UV-treat 50ml conical tube. Repeat until all of the solution has been dispensed into conical tubes. Add 2.5 ml of filtered KCl to each tube of a poly-A solution, and 22.5 ml of 100% isopropanol. Cap each tube and mix by inversion. Centrifuge tubes at 3000 rpm for 10 minutes. Carefully remove supernatant by pouring off. Let sit right side up for 2 minutes, then pipette out any remaining isopropanol. Let sit inverted on a kimwipe or blotting paper equivalent for 2-3 minutes to generate a dried pellet. The dried pellet from each tube may be resuspended in 2 ml dilution buffer and then all the samples may be pooled.

### Example 2

### Comparison to Prior Art Method

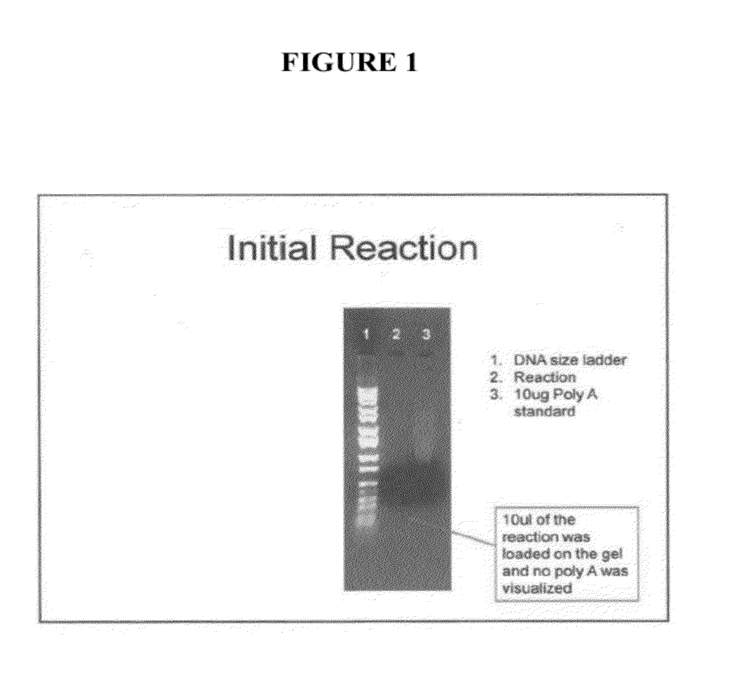

This Example describes polyadenylic acid synthesis methods from the prior art (Jones and Bibb, J. of Bact., 1996, 178(14): 4281-4288, herein after “Jones,” which is herein incorporated by reference) in comparison to exemplary methods of the present invention. The initial reaction conditions from Jones include: 54.3 mM Adenosine diphosphate (ADP), 50 mM Magnesium Chloride, 50 mM Tris pH9.1, and 15 ul (17.1 units) polynucleotide phosphorylase, in a 16.5 ml reaction at 42 degrees Celsius overnight. This reaction produced no poly-A (as visualized using gel electrophoresis), as shown in FIG. 1.

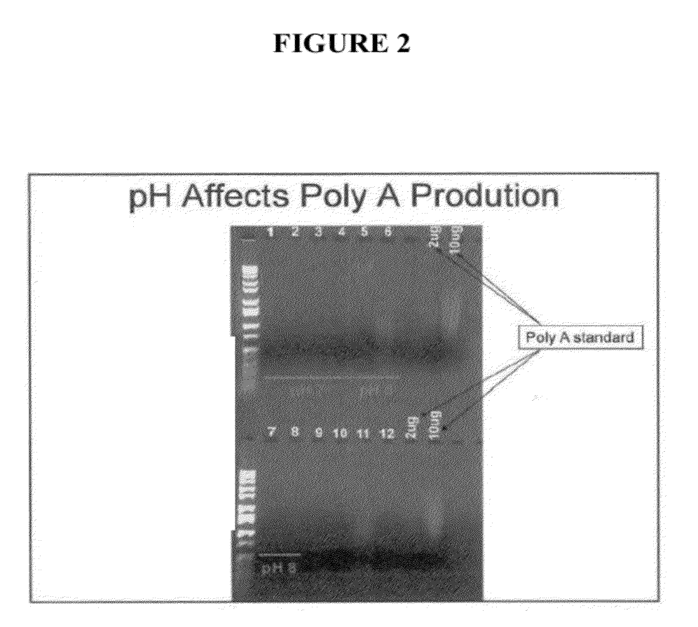

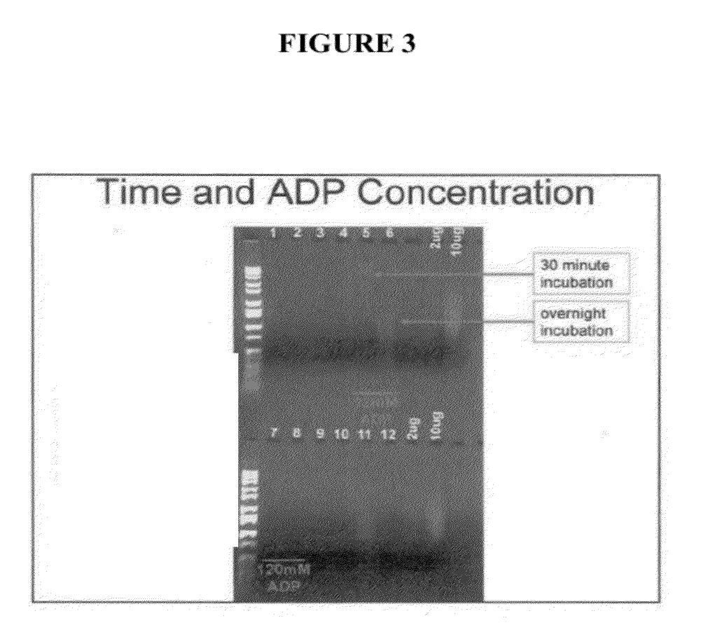

Following these results, variations of ADP concentrations, pH, and incubation time were changed from these initial conditions to see if this would positively affect the production of poly A. After gel electrophoresis of these results it was determined that there was poly A produced in reactions that were buffered to pH 8.0 (See FIG. 2). This however was inhibited by high concentrations of ADP (see FIG. 3). Also it appeared that short times of incubation produced high molecular weight poly A and long incubation times produced lower molecular weight poly A at pH 8.0 (see FIG. 3). It is noted that this would be consistent if there was degradation over time taking place.

### Example 3

### Optimization of Reaction Conditions

This Example describes further optimization of reactions conditions from those shown to produce polyA in Example 2. After testing various parameters, an exemplary optimized set of reactions conditions was determined. These reaction conditions included a concentration of 23.2 mM ADP, 50 mM tris pH 8.0, 10 mM magnesium chloride and 0.42 units/ml polynucleotide phosphorylase. This reaction was left at 42 degrees Celsius for 115 hours. Under these conditions 1 gram of poly A was produced in 500 ml of reaction volume.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising or consisting essentially of:
a) polynucleotide phosphorylase;
b) adenosine diphosphate (ADP) present at a concentration between 5.0 mM and 100 mM;
c) a buffering agent with a pH between 7.3 and 8.7; and
d) a divalent metal cation.

2. The composition of claim 1, wherein said composition is free of detectable contaminating nucleic acid.

3. The composition of claim 1, wherein said pH is between 7.7 and 8.3.

4. The composition of claim 1, wherein said pH is between 7.9 and 8.1.

5. The composition of claim 1, wherein said ADP is present at a concentration between 10.0 mM and 75 mM.

6. The composition of claim 1, wherein said ADP is present at a concentration between 20.0 mM and 50 mM.

7. The composition of claim 1, wherein said divalent metal cation is MgCl₂.

8. A method of making polyadenylic acid comprising:
a) combining polynucleotide phosphorylase, adenosine diphosphate (ADP), a buffering agent with pH between 7.4 and 8.6, and a divalent metal cation, to generate a mixture, wherein said ADP is present in said mixture at a concentration between 5.0 mM and 100 mM; and
b) incubating said mixture under conditions such that a composition comprising polyadenylic acid is generated.

9. The method of claim 8, wherein said incubating is conducted for 30 minutes to 150 hours.

10. The method of claim 8, wherein said incubating is conducted for 100 hours to 125 hours.

11. The method of claim 8, wherein said incubating is conducted at a temperature between 30 and 50 degrees Celsius.

12. The method of claim 8, wherein said incubating is conducted at a temperature between 38 and 46 degrees Celsius.

13. The method of claim 8, wherein said composition is free of detectable contaminating nucleic acid.

14. The method of claim 8, wherein said pH is between 7.7 and 8.3.

15. The method of claim 8, wherein said pH is between 7.9 and 8.1.

16. The method of claim 8, wherein said ADP is present at a concentration between 10.0 mM and 75 mM.

17. The method of claim 8, wherein said ADP is present at a concentration between 20.0 mM and 50 mM.

18. The method of claim 1, wherein said divalent metal cation is MgCl₂.

19. A system comprising, or consisting essentially of, or consisting of, the following components:
a) polynucleotide phosphorylase;
b) adenosine diphosphate (ADP) present at a concentration between 5.0 mM and 100 mM;
c) a buffering agent with a pH between 7.4 and 8.6; and
d) a divalent metal cation.

20. The system of claim 19, wherein said divalent metal cation is MgCl₂.
